(19) United States
(12) Patent Application Publication
(10) Pub. No.: US 2025/0381037 A1
(43) Pub. Date: Dec. 18, 2025

(54) BIONIC ACETABULAR PROSTHESIS

(71) Applicant: HEFEI LONGSHORE TECH CO., LTD, Hefei City (CN)

(72) Inventors: Luoping ZHOU, Hefei City (CN); Lifeng ZHANG, Hefei City (CN)

(21) Appl. No.: 19/080,479

(22) Filed: Mar. 14, 2025

Related U.S. Application Data
(63) Continuation-in-part of application No. PCT/CN2024/125184, filed on Oct. 16, 2024.

(30) Foreign Application Priority Data
Jun. 17, 2024 (CN) 202410775002.1

(51) Int. Cl. A61F 2/34; A61F 2/30

(52) U.S. Cl. CPC A61F 2/34; A61F 2002/30158; A61F 2002/30827; A61F 2002/30881; A61F 2002/30897; A61F 2002/343; A61F 2002/349

(57) ABSTRACT

A bionic acetabular prosthesis has a spherically cap-shaped cup body. The body is inwardly concave at the bottom to form a hollow cavity, and has on its outer wall surface, annular collars, each having a trapezoidal cross-section with a larger base portion and a smaller top portion. The collars are provided with circumferentially-spaced notches, having a recessed depth smaller than or equal to a raised height of the collars. Slopes of opposite side faces of the cross-sectionally trapezoidal collars on the cup body avoid the formation of defects in the surface of the hollow cavity, ensuring good quality of the resulting acetabular prosthesis. In addition, notches may be arbitrarily formed in the collars. These notches interact with bone cement to restrict circumferential rotation of the cup body. Gently sloped surfaces of adjacent collars join and define annular grooves, which contain more bone cement, stabilizing the cup body.

### RELATED APPLICATIONS

This application is a continuation-in-part (CIP) application claiming benefit of PCT/CN2024/125184 filed on Oct. 16, 2024, which claims priority to Chinese Patent Application No. 202410775002.1 filed on Jun. 17, 2024, the disclosures of which are incorporated herein in their entirety by reference.

### BACKGROUND

The present invention relates to the field of joint prostheses and, in particular, to a bionic acetabular prosthesis.

### DESCRIPTION OF THE PRIOR ART

Artificial hip joint replacement is a therapy involving the replacement of a diseased hip joint with acetabular and femoral prostheses. The acetabular prosthesis can be anchored to the acetabulum with bone cement, which serves as a bonding interface. This approach has found extensive use thanks to its advantage of allowing fast postoperative recovery.

Chinese Patent No. CN209253232U (hereinafter identified as “Document 1”) discloses a cement-based acetabular prosthesis comprising a cup body in the form of a hemispherical structure and defining an accommodating cavity therein. Transverse and vertical grooves are formed in an outer surface of the cup body, and clamping rings are locked in the transverse grooves. The transverse grooves are parallel to one another, and the vertical grooves extend along vertical planes of symmetry of the cup body. All the vertical grooves converge at one end on the topmost transverse groove around an apex of the cup body. Each pair of symmetric vertical grooves extends along a single vertical plane, which bisects the cup body into equal halves. The cement-based acetabular prosthesis is implanted into the acetabular fossa of a patient after bone cement is filled therein. The transverse grooves on the acetabular prosthesis prevent axial displacement of the prosthesis, and the vertical grooves prevent its rotation within the acetabular fossa. The clamping rings are made of a metal material. These metal rings can be visualized in X-ray images, helping a surgeon determine the position of the cement-based acetabular prosthesis in the patient's body. Chinese Patent No. CN213250057U (hereinafter identified as “Document 2”) discloses an acetabular cup with a radiopaque wire, comprising an acetabular cup body, which is a unitary one-piece part made of an ultra-high molecular weight polyethylene (UHMWPE) material. The acetabular cup body comprises a lower hemispherical portion and an upper tapered portion on the hemispherical portion. The tapered portion has a smaller diameter than the hemispherical portion, in order to restrict the ball-shaped end of the femoral head from luxation. In the exterior of the hemispherical portion are formed multiple parallel annular grooves, which are equidistantly spaced from the pole to the equator, and vertical grooves connecting the pole and the equator. The radiopaque wire is provided in the annular groove with the greatest diameter.

As shown in the drawings accompanying the specifications of the Documents 1 and 2, the transverse and vertical grooves in the outer surface of each of those conventional hemispherical acetabular prostheses define a number of mesas much greater than the openings of the vertical and transverse grooves measure at their top edges. This design makes it difficult for bone cement to access cavities of the grooves, so stable anchoring of the acetabular prostheses cannot be ensured. As another consequence, there are steep wall thickness variations around the transverse and vertical grooves, which necessitates a large wall thickness of the acetabular prostheses for preventing undesirable surface unevenness that may otherwise arise from inner surface shrinkage and deformation during manufacturing of the acetabular prostheses. This, however, means a reduced range of motion of the hip joint, when compared with an acetabular prosthesis with the same outer diameter and a smaller wall thickness.

### SUMMARY

It is an object of the present invention to provide a bionic acetabular prosthesis, which is easy to manufacture and can be anchored with desired stability.

The above object is attained by a bionic acetabular prosthesis of the present invention, which comprises a cup body generally in the form of a spherical cap. The cup body is inwardly concave at the bottom to form a hollow cavity. An inner wall surface of the hollow cavity comprises a spherical cap surface matching an outer diameter of a ball head prosthesis. The cup body is provided with, on its outer wall surface, a number of generally annular collars each having a cross-section generally in the shape of a trapezoid having a larger base portion and a smaller top portion. Adjacent collars define annular grooves therebetween at joints of their base portions. The collars are provided with notches thereon spaced at circumferential intervals, which have a recessed depth smaller than or equal to a raised height of the collars.

The present invention offers the following benefits over the prior art: based on a material, of which the cup body is made, slopes of opposite side faces of the generally cross-sectionally trapezoidal collars on the cup body can be configured to avoid the formation of defects in the surface of the hollow cavity, ensuring good quality of the resulting acetabular prosthesis; and notches may be arbitrarily formed in the collars, as required, which can interact with bone cement to restrict circumferential rotation of the cup body; and gently sloped surfaces of adjacent collar join and together define annular grooves with large openings, which can contain more bone cement when in use, stabilizing the cup body in its heightwise direction after it is anchored.

### BRIEF DESCRIPTION OF THE DRAWINGS

The drawings accompanying this specification, as well as reference numerals therein, are described briefly below, in which:

FIGS. 1 and 2 are schematic perspective views of a first embodiment;

FIG. 3 is a top view of the first embodiment;

FIG. 4 is a cross-sectional view taken along B-B of FIG. 3;

FIG. 5 is a cross-sectional view taken along A-A of FIG. 3;

FIG. 6 is a schematic perspective view of a second embodiment;

FIG. 7 is a front view of a second embodiment;

FIG. 8 is a cross-sectional view taken along C-C of FIG. 7; and

FIG. 9 is a cross-sectional view of a second embodiment taken along a plane passing through a center line but not through any notch.

In these figures, 10 denotes a cup body; 11, a hollow cavity; 111, a spherical cap surface; 112, a tapered surface; 12, a collar; 121, a truncated surface; 122, an upper surface; 122a, a collar flat surface portion; 13, a notch; 14, a locating mesa; 141, a top surface; 15, a flange ring; 151, a flange ring outer surface; 152, an upper surface; 152a, a flange ring flat surface portion, 16, an annular groove; and 20, a radiopaque wire.

### DETAILED DESCRIPTION

Specific embodiments of the present invention are described in greater detail below, by way of example, with reference to the accompanying drawings.

### Example 1

A bionic acetabular prosthesis comprises a cup body 10 generally in the form of a spherical cap. The cup body 10 is inwardly concave at the bottom to form a hollow cavity 11. An inner wall surface of the hollow cavity 11 includes a spherical cap surface 111 for mating with a ball head prosthesis in a manner allowing relative rotation. In order to ensure the stability and reliability of the mating, the inner wall surface of the spherical cap surface 111 is desired to be smooth and have a diameter coinciding with an outer diameter of the ball head prosthesis.

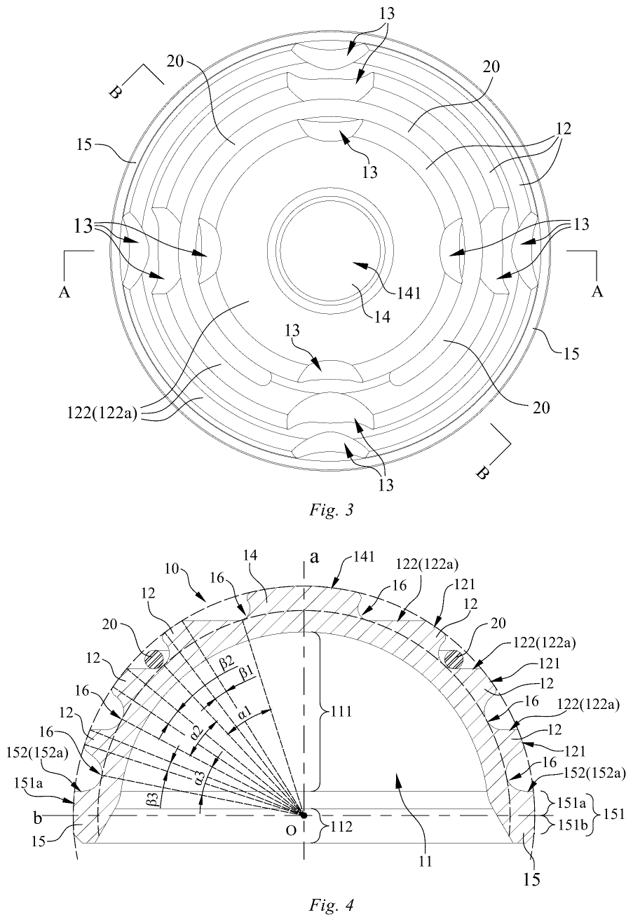

In order for stable anchoring of the acetabular prosthesis to be achieved, the cup body 10 is provided with, on its outer wall surface, a number of generally annular collars 12. In use, bone cement can fill cavities of annular grooves 16 and harden therein, restricting the cup body 10 from displacement along its center line a. As shown in FIG. 1, by “annular”, it is intended to mean that the collars 12 resemble closed rings scattered on the outer wall of the cup body 10. These collar rings are continuous without any gaps, additionally ensuring that the cup body 10 is more stably and more reliably restricted by the bone cement from displacement along its center line a. As shown in FIG. 4, each collar 12 has a cross-section taken in a direction perpendicular to its annular center line, which is generally in the shape of a trapezoid having a larger base portion and a smaller top portion. A wall thickness of the cup body 10 gradually increases from either side of each collar 12 toward its center. This can effectively avoid quality defects in the inner wall surface of the hollow cavity 11, which may be otherwise caused if the wall thickness has too steep variations. Adjacent collars 12 join at their shorter base portions, which define the annular grooves 16 together with taller top portions of the collars 12.

As shown FIG. 3, the collars define notches 13 therein, which are circumferentially spaced around the collars. In use, bone cement can fill recessed cavities of the notches 13 and harden therein, restricting the cup body 10 from rotational displacement about its center line a. In the present embodiment, a recessed depth of the notches 13 is smaller than or equal to a raised height of the collars 12. That is, a minimum distance between inner concave surfaces of the notches 13 and a center of the spherical cap surface 111 is greater than a minimum distance between convex surfaces of the collars 12 and the center of the spherical cap surface 111. With this arrangement, although the raised collars 12 have different heights at different portions, they are still continuous structures. In addition, given the collars 12 will not cause quality defects in the inner wall surface of the hollow cavity 11, it is a matter of course that the notches 13, the inner wall surfaces of which face away from the hollow cavity 11, and at which the wall thickness of the cup body 10 varies less steeply than at the collars 12, will also not cause quality defects in the inner wall surface of the hollow cavity 11. In other words, during the design of the prosthesis, once the shape and contour of the collars 12 have been determined, based on the material selected, to ensure desirable quality of the spherical cap surface 111, more freedom is left for the arrangement of the notches 13.

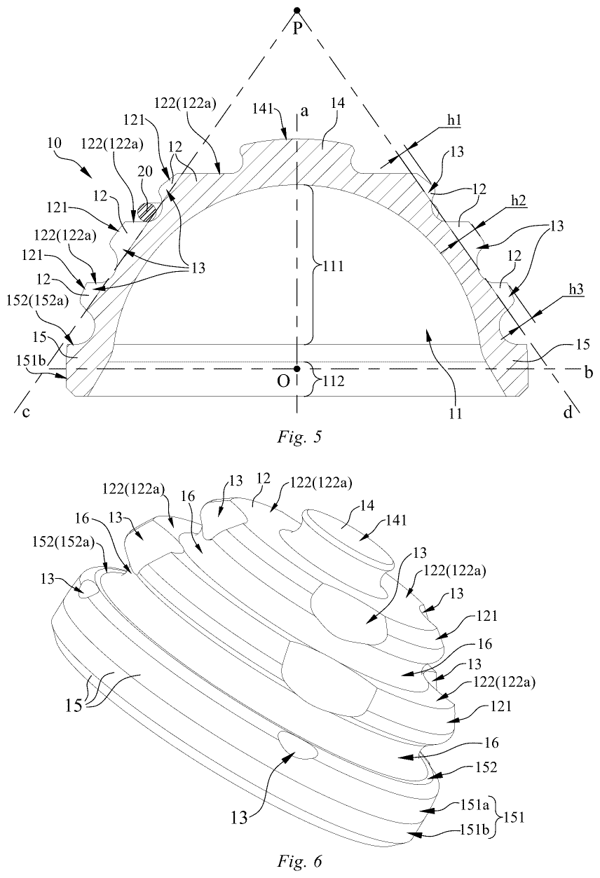

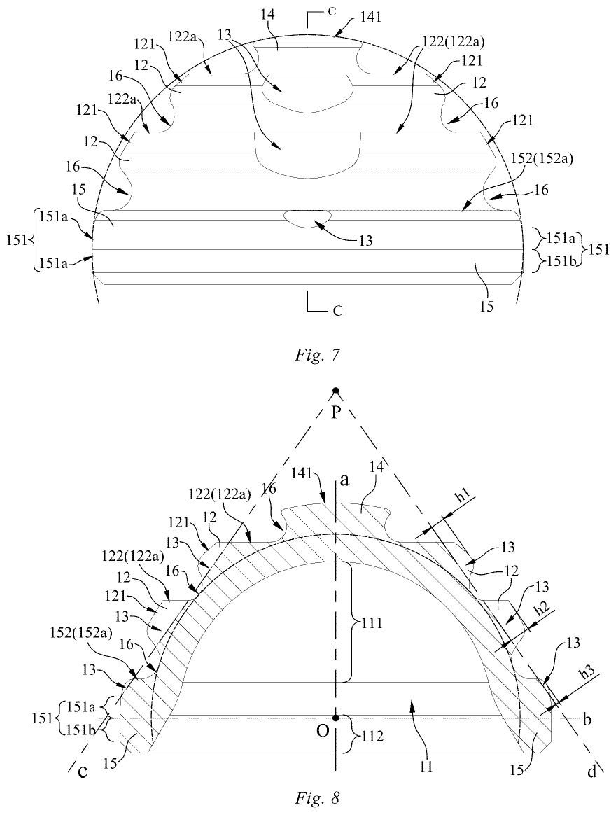

For each notch 13, a depth is defined at the deepest point of the inner concave surface in a radially direction of the cup body 10, and a width is defined as a maximum distance between two ends of the inner concave surface measured in a circumferential direction of the cup body 10. A greater depth and/or width allows the notches 13 to interact with bone cement to better restrict rotation of the cup body 10 and to enable adjustments to be more easily made in an orientation of the cup body 10, in which it mates with the acetabular fossa. However, an excessively large depth or width of notches 13 on a lower portion of the cup body 10 may lead to leakage of bone cement around an opening of the cup body 10. Moreover, since one or more collars 12 on an upper portion of the cup body 10 each have a small diameter of the annular center line, an excessively large depth or width of notches 13 in this or these collars may make its or their effective lengths too small to ensure desired resistance to axial luxation. In order to avoid this, as shown in FIG. 5, in the present embodiment, notches 13 arranged at the middle of the cup body 10 in a heightwise direction thereof have a greater depth than notches 13 above and below them, i.e., h2>h1 and h2>h3, as shown in the figure. Meanwhile, in the present embodiment, the notches 13 arranged at the middle of the cup body 10 in the heightwise direction have a greater width than those above and below them. In other embodiments, the middle notches 13 may only have a greater depth, or a greater width, than those above and below them.

Further, the inner concave surfaces notches 13 vertically aligned at the same side of the cup body 10 may be located in a single cylindrical plane. This can facilitate the positioning of a machining tool or mold. With this in mind, in order to ensure that the notches 13 have a recessed depth greater than a raised height of the collars 12, as shown in FIG. 5, the notches 13 are arranged in symmetry with respect to the center line a of the cup body so that the inner concave surfaces of notches 13 on opposite sides of the center line a of the cup body have their center lines of c, d intersecting a point P located above the cup body 10. With this arrangement, once a positional relationship of the center lines c, d of the inner concave surfaces and the center line a of the cup body is determined, the recessed depth of the notches 13 can be simply determined. In the present embodiment, as shown in FIG. 3, four sets of vertically-aligned notches 13 are arranged at equal angular intervals circumferentially around the cup body 10, and the center lines of the inner concave surfaces of the notches 13 intersect at a single point to facilitate the positioning of a machining tool or mold.

As shown in FIG. 4, the collars 12 include truncated surfaces 121 located away from the hollow cavity 11. The truncated surfaces 121 are convex top surfaces of the collars 12 on the cup body 10 and extend in a single spherical plane. A top portion of the cup body 10 is provided with an upwardly raised locating mesa 14 with a top surface 141 extending in the single spherical plane as the truncated surfaces 121 of the collars 12. The locating mesa 14 is smaller at the bottom and greater at the top, resembling a mushroom. A base portion of the locating mesa 14 is inwardly recessed to also define an annular groove 16. The cup body 10 is provided with a flange ring 15 running around its circumference at the bottom, and upper surface portion 151a of a flange ring outer surface 151 extends in the single spherical plane as the truncated surfaces 121. A bottom edge of the upper surface portion extends downwardly in a direction tangential to the spherical plane in which the upper surface portion is located to form a cylindrical lower surface portion.

As shown in FIG. 4, except for a portion around the opening, the majority of an outer surface of the cup body 10 extends in a single spherical plane. This can facilitate adjusting the cup body 10 to an orientation suitable for its anchorage. The locating mesa 14 can ensure that the cup body 10 is anchored so that its center line a is accurately oriented in a desired direction. That is, it can be ensured that the cup body 10 is anchored in the acetabular fossa accurately with respect to a depthwise direction thereof.

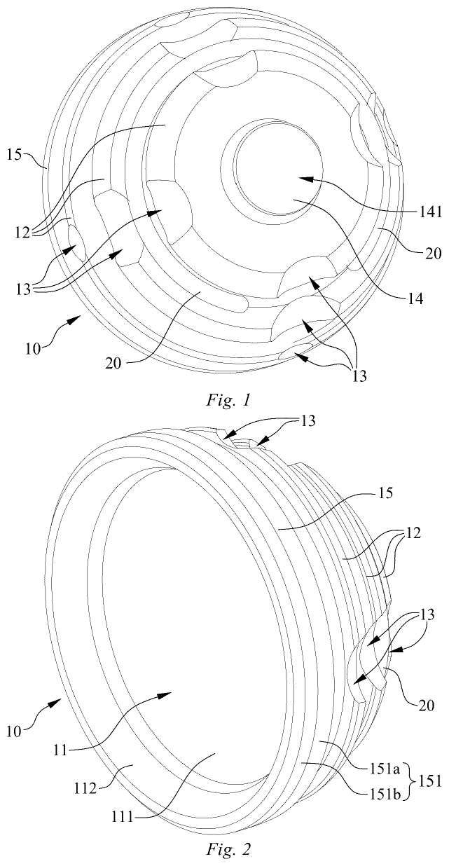

Upper surfaces 122 of the collars 12 include annular planar collar flat surface portions 122a, the planes in which each of the collar flat surface portions is located are parallel. This facilitates withdrawal of a tool or mold used for manufacturing in a direction parallel to the collar flat surface portions 122a. In the present embodiment, the planes in which the annular center lines of collars 12, as well as the collar flat surface portions 122a thereof are located, are perpendicular to the center line a of the cup body. Likewise, to facilitate manufacturing and machining, as shown in FIGS. 2 and 4, the plane in which the annular center line of the flange ring 15 is located is perpendicular to the center line a of the cup body, an upper surface 152 of the flange ring 15 includes an annular planar flange ring flat surface portion 152a, which is also perpendicular to the center line a of the cup body. The cup body 10 also has an open end face, which is a flat surface also perpendicular to the center line a of the cup body. With this arrangement, a tool or mold used to manufacture the cup body 10 can be withdrawn in a direction perpendicular to the center line a of the cup body.

In order to ensure good appearance quality of the cup body 10, in the present embodiment, the collar flat surface portions 122a join, along their outer edges, the truncated surfaces 121 at obtuse angles. With this arrangement, the formation of burrs and the like, which may affect the appearance quality, can be inhibited. Therefore, the collar flat surface portions 122a directly join the truncated surfaces 121 along the outer edges of the collar flat surface portions 122a. In other embodiments, the joints of the collar flat surface portions 122a and the truncated surfaces 121 along the outer edges of the collar flat surface portions 122a may be chamfered. Each collar flat surface portion 122a joins, along its inner edge, the overlying collar 12 or locating mesa 14 at an acute angle. In order to ensure good appearance quality of the cup body 10, the inner edge of each collar flat surface portion 122a may be joined by an inwardly curved smooth transition surface to the overlying collar 12 or locating mesa 14. Likewise, in the present embodiment, the flange ring flat surface portion 152a joins, along its outer edge, the flange ring outer surface 151 at an almost right angle. In order to ensure good appearance quality at the joint, the flange ring flat surface portion 152a may be chamfered or filleted along its outer edge and then join the flange ring outer surface 151. In other embodiments, the outer edge of the flange ring flat surface portion 152a may directly join the flange ring outer surface 151. The flange ring flat surface portion 152a joins the overlying collar 12 at an acute angle. In order to ensure good appearance quality of the cup body 10, the inner edge of the flange ring flat surface portion 152a may be joined by an inwardly curved smooth transition surface to the overlying collar 12.

In the present embodiment, the cup body 10 is made of a polyetheretherketone (PEEK) material. Compared with commonly used polyethylene (PE) materials, the PEEK material exhibits higher strength and allows the cup body 10 to have an additionally reduced wall thickness. In order to enable reliable shape retention of the cup body 10 and the stability of its inner spherical cap surface 111, the wall thickness is desired to increase with the size of the cup body 10. In preferred embodiments, if an outer diameter of the cup body 10 is less than 17 mm, the wall thickness in the range of 1 mm to 2 mm. If the outer diameter of the cup body 10 is greater than or equal to 17 mm and less than 25 mm, then the wall thickness ranges from 1.5 mm to 3.5 mm. If the outer diameter of the cup body 10 is greater than 25 mm, then the wall thickness ranges from 2 mm to 5 mm. Here, the outer diameter refers to an outer diameter of an outer contour of the cup body 10 and is used as an indicator of its size. In the present embodiment, the outer diameter of the cup body 10 refers to a diameter of the spherical plane in which the truncated surfaces 121 of the collars extend. In order to ensure that the collars 12 can effectively interact with bone cement to restrict the cup body, the raised height of the collars 12 is desired to be greater than or equal to 0.5 mm. At the same time, in order to obtain a thin-wall cup body 10, which provides enhanced resistance of the acetabular prosthesis to luxation, the raised height of the collars 12 is desired to be less than half the wall thickness of the cup body 10.

In conventional acetabular prostheses, a cup body 10 with an outer diameter less than 17 mm usually has a wall thickness of at least 3 mm. In contrast, according to the present embodiment, such a cup body 10 has a wall thickness of at most 2 mm. This means that, at the given outer diameter of an acetabular prosthesis to be used with a ball head prosthesis in a hip joint replacement procedure, this embodiment allows the ball head prosthesis to have an outer diameter that is at least 1 mm greater, and hence to more displace at least 1 mm before it can dislodge, when compared with the prior art. Thus, the risk of luxation of the ball head prosthesis is significantly reduced, and the hip joint is allowed to move within a wider range.

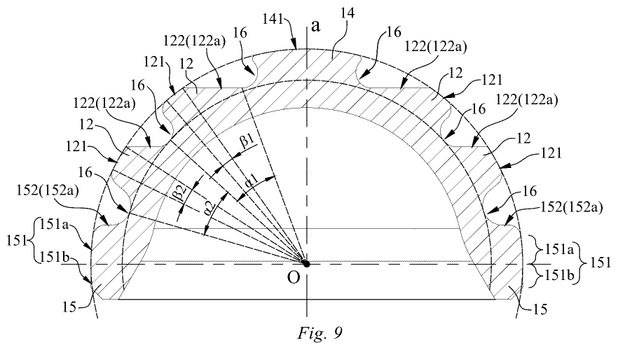

In order to enable the spherical cap surface 111 to have desirable quality at a small wall thickness, in preferred embodiments, the shape and contour of the collars 12 is configured so that an angle α defined by the base portion of the topmost collar 12 is 3-7 times an angle β defined by the top portion of the collar, and an angle α defined by the base portion of each other collar 12 is 3-5 times an angle β defined by the top portion of the collar. That is, in FIGS. 4 and 9, ∠α₁ is 3-7 times ∠β₁, and ∠α₂ is 3-5 times ∠β₂.

As shown in FIG. 4, in each collar, the angle α defined by the base portion is an angle of an arc spanned by the base portion, and the angle β defined by the top portion is an angle of an arc spanned by the top portion. As shown also in FIG. 4, the locating mesa 14 is raised upwards from the middle the upper surface 122 of the topmost collar 12, and the collar flat surface portion 122a of the upper surface 122 of the topmost collar 12 joins the base portion of the centrally located locating mesa 14. Moreover, this collar flat surface portion 122a has a significantly greater difference between its inner and outer diameters than any other collar flat surface portion 122a. Therefore, the angle of the base portion of the topmost collar 12 is greater than the angle of the base portion of any other collar 12.

Furthermore, more collars 12 mean more annular grooves 16, which enable better restriction of the cup body 10 along its center line a. Accordingly, the top portions may define an angle β of 3-9°, so at least two collars 12, and hence at least two annular grooves 16, may be formed on the outer surface of the cup body 10.

In the present embodiment, as shown in FIGS. 4 and 5, the spherical cap surface 111 may be arranged at an upper portion of the hollow cavity 11, and the inner wall surface may further include a lower tapered surface 112. The spherical cap surface 111 and the tapered surface 112 may be joined by a curved smooth transition surface. The spherical cap surface 111 defines a height less than its radius. That is, its center O is encompassed within a space delimited by the tapered surface 112. With this arrangement, during use, a ball head prosthesis may be placed with its center being located within the hollow cavity 11 of the cup body 10 and thus has to displace a distance greater than its own radius before it can move out of the cup body 10, reducing the risk of luxation. The ball head prosthesis may have a neck portion joined to a femoral stem, leaving a chance for the opening edge of the cup body 10 to interfere with the neck portion. However, the flange ring 15 arranged around the opening edge of the cup body 10 thickens the cup body 10 at the location, and the inner tapered surface 112 of the flange ring 15 can avoid the open end of the hollow cavity 11 from interfering with the neck portion and additionally expands the range of motion of the ball head prosthesis.

As shown in FIGS. 1 and 3, during use, a metal radiopaque wire 20 may be received within one of an annular groove 16, which enables an orientation of the cup body 10, in which it is anchored, to be determined during a medical examination. In the present embodiment, as shown in FIG. 3, the radiopaque wire 20 may be a generally C-shaped open ring.

### Example 2

A second embodiment differs from the first embodiment in that notches 13 are provided at circumferential intervals in an upper edge of the flange ring 15. In order to prevent overflow of bone cement along the outer circumference of the cup body 10, in the present embodiment, the notches 13 join, at the bottom, the upper surface portion 151a of the flange ring outer surface. That is, the notches 13 are formed above the lower surface portion 151b of the outer surface. In the present embodiment, in order to facilitate machining, the notches 13 in the flange ring 15 are located on the same cylindrical planes as the notches 13 in the collars 12.

While particular embodiments of the present bionic acetabular prosthesis have been described herein, it will be appreciated by those skilled in the art that changes and modifications may be made thereto without departing from the invention in its broader aspects and as set forth in the following claims.

1. A bionic acetabular prosthesis, comprising: a cup body generally in the form of a spherical cap, the cup body inwardly concave at the bottom to form a hollow cavity, an inner wall surface of the hollow cavity comprising a spherical cap surface matching an outer diameter of a ball head prosthesis; wherein the cup body is provided with, on its outer wall surface, a number of generally annular collars each having a cross-section generally in the shape of a trapezoid having a larger base portion and a smaller top portion; adjacent collars defining annular grooves therebetween at joints of their base portions; the collars provided with notches thereon spaced at circumferential intervals, the notches having a recessed depth smaller than or equal to a raised height of the collars.

2. The bionic acetabular prosthesis according to claim 1, wherein the collars have truncated surfaces away from the hollow cavity, which extend in a single spherical plane, wherein:
a top portion of the cup body is provided with an upwardly raised locating mesa, the locating mesa having a mushroom-like shape smaller at the bottom and greater at the top, the locating mesa having a top surface extending in the single spherical plane as the truncated surfaces of the collars;
the cup body is provided with a flange ring running around its circumference at the bottom, a flange ring outer surface having an upper surface portion extending in the single spherical plane as the truncated surfaces, a bottom edge of the upper surface portion extending downwardly in a direction tangential to the spherical plane in which the upper surface portion is located to form a cylindrical lower surface portion.

3. The bionic acetabular prosthesis according to claim 2, wherein upper surfaces of the collars comprise annular planar collar flat surface portions, the planes in which each of the collar flat surface portions is located are parallel, wherein:
outer edges of the collar flat surface portions are directly joined to the truncated surfaces, or are chamfered/filleted and then joined to the truncated surfaces, and an inner edge of each collar flat surface portion is joined to the overlying collar or locating mesa by an inwardly curved smooth transition surface; and
the planes in which annular center lines of the collars are located are perpendicular to a center line (a) of the cup body, and the planes in which the collar flat surface portions are located are perpendicular to the center line (a) of the cup body.

4. The bionic acetabular prosthesis according to claim 2, wherein an angle α of the base portion of a topmost one of the collars is 3-7 times an angle β of the top portion of the collar, and an angle α of the base portion of each other collar is 3-5 times an angle β of the top portion of the collar, wherein in each collar, the angle α of the base portion is defined as an angle of an arc spanned by the base portion, and the angle β of the top portion is defined as an angle of an arc spanned by the top portion and is 3° to 9°.

5. The bionic acetabular prosthesis according to claim 2, wherein notches are provided at an upper edge of the flange ring spaced at circumferential intervals and joined at the bottom to the upper surface portion.

6. The bionic acetabular prosthesis according to claim 2, wherein the plane in which an annular center line of the flange ring is located is perpendicular to the center line (a) of the cup body, wherein an upper surface of the flange ring comprises an annular planar flange ring flat surface portion perpendicular to the center line (a) of the cup body; an outer edge of the flange ring flat surface portion is directly joined to the flange ring outer surface, or is chamfered/filleted and then joined to the flange ring outer surface; and an inner edge of the flange ring flat surface portion is joined to the overlying collar by an inwardly curved smooth transition surface.

7. The bionic acetabular prosthesis according to claim 1, wherein ones of the notches located at the middle of the cup body in a heightwise direction thereof have a depth greater than a depth of the notches located above or below them, and/or in that the notches located at the middle of the cup body in the heightwise direction thereof have a width greater than a width of the notches located above or below them.

8. The bionic acetabular prosthesis according to claim 7, wherein inner concave surfaces of ones of the notches vertically aligned at the same side of the cup body is located in a single cylindrical plane.

9. The bionic acetabular prosthesis according to claim 8, wherein the notches are arranged in symmetry with respect to the center line of the cup body, wherein center lines of the inner concave surfaces of ones of the notches arranged at opposite sides of the center line of the cup body intersect above the cup body.

10. The bionic acetabular prosthesis according to claim 1, wherein an open end face of the cup body is a flat surface perpendicular to a center line of the cup body, wherein the spherical cap surface is arranged at an upper portion of the hollow cavity, and the inner wall surface of the hollow cavity further comprises a lower tapered surface; the spherical cap surface and the tapered surface are joined by a curved smooth transition surface; a height of the spherical cap surface is less than its radius; a radiopaque wire made of a metal is embedded in one of the annular grooves; and the radiopaque wire is generally C-shaped.

11. The bionic acetabular prosthesis according to claim 1, wherein the cup body is made of a polyetheretherketone material and has a wall thickness in the range of 1 mm to 2 mm if its outer diameter is less than 17 mm, or of 1.5 mm to 3.5 mm if its outer diameter is greater than or equal to 17 mm and smaller than or equal to 25 mm, or of 2 mm to 5 mm if its outer diameter is greater than 25 mm, wherein
the raised height of the collars is greater than or equal to 0.5 mm and less than half the wall thickness of the cup body.

12. The bionic acetabular prosthesis according to claim 2, wherein ones of the notches located at the middle of the cup body in a heightwise direction thereof have a depth greater than a depth of the notches located above or below them, and/or in that the notches located at the middle of the cup body in the heightwise direction thereof have a width greater than a width of the notches located above or below them.

13. The bionic acetabular prosthesis according to claim 3, wherein ones of the notches located at the middle of the cup body in a heightwise direction thereof have a depth greater than a depth of the notches located above or below them, and/or in that the notches located at the middle of the cup body in the heightwise direction thereof have a width greater than a width of the notches located above or below them.

14. The bionic acetabular prosthesis according to claim 4, wherein ones of the notches located at the middle of the cup body in a heightwise direction thereof have a depth greater than a depth of the notches located above or below them, and/or in that the notches located at the middle of the cup body in the heightwise direction thereof have a width greater than a width of the notches located above or below them.

15. The bionic acetabular prosthesis according to claim 5, wherein ones of the notches located at the middle of the cup body in a heightwise direction thereof have a depth greater than a depth of the notches located above or below them, and/or in that the notches located at the middle of the cup body in the heightwise direction thereof have a width greater than a width of the notches located above or below them.

16. The bionic acetabular prosthesis according to claim 6, wherein ones of the notches located at the middle of the cup body in a heightwise direction thereof have a depth greater than a depth of the notches located above or below them, and/or in that the notches located at the middle of the cup body in the heightwise direction thereof have a width greater than a width of the notches located above or below them.
